(19) United States
(12) Patent Application Publication
(10) Pub. No.: US 2009/0318468 A1
(43) Pub. Date: Dec. 24, 2009

(54) PYRAZOLE COMPOUNDS 436

(75) Inventors: David Buttar, Macclesfield (GB); Maria-Elena Theoclitou, Macclesfield (GB); Andrew Peter Thomas, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/487,838

(22) Filed: Jun. 19, 2009

Related U.S. Application Data
(60) Provisional application No. 61/073,883, filed on Jun. 19, 2008.

(51) Int. Cl. A61K 31/496; C07D 403/12; A61P 35/00

(52) U.S. Cl. 514/254.05; 544/371

(57) ABSTRACT

There is provided novel compounds of formula (Ia) or (Ib):

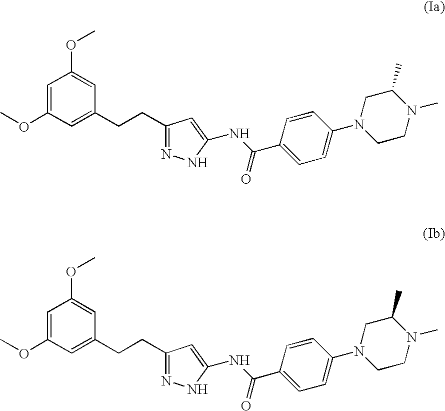

or pharmaceutically acceptable salsts thereof, processes for their preparation, pharmaceutical compositions containing them and their use in therapy.

This application claims the benefit under under 35 U.S.C. §119(e) of Application No. U.S. 61/073,883 filed on 19 Jun. 2008.

The present invention relates to pyrazole compounds, a process for their preparation, pharmaceutical compositions containing them, a process for preparing the pharmaceutical compositions, and their use in therapy.

Protein kinases are a class of proteins (enzymes) that regulate a variety of cellular functions. This is accomplished by the phosphorylation of specific amino acids on protein substrates resulting in conformational alteration of the substrate protein. The conformational change modulates the activity of the substrate or its ability to interact with other binding partners. The enzyme activity of the protein kinase refers to the rate at which the kinase adds phosphate groups to a substrate. It can be measured, for example, by determining the amount of a substrate that is converted to a product as a function of time. Phosphorylation of a substrate occurs at the active-site of a protein kinase.

Tyrosine kinases are a subset of protein kinases that catalyze the transfer of the terminal phosphate of adenosine triphosphate (ATP) to tyrosine residues on protein substrates. These kinases play an important part in the propagation of growth factor signal transduction that leads to cellular proliferation, differentiation and migration.

Fibroblast growth factor (FGF) has been recognized as an important mediator of many physiological processes, such as morphogenesis during development and angiogenesis. There are currently over 25 known members of the FGF family. The fibroblast growth factor receptor (FGFR) family consists of four members with each composed of an extracellular ligand binding domain, a single transmembrane domain and an intracellular cytoplasmic protein tyrosine kinase domain. Upon stimulation with FGF, FGFRs undergo dimerisation and transphosphorylation, which results in receptor activation. Receptor activation is sufficient for the recruitment and activation of specific downstream signalling partners that participate in the regulation of diverse process such as cell growth, cell metabolism and cell survival (Reviewed in Eswarakumar, V. P. et. al., Cytokine & Growth Factor Reviews 2005, 16, p 139-149). Consequently, FGF and FGFRs have the potential to initiate and/or promote tumorigenesis.

There is now considerable evidence directly linking FGF signalling to human cancer. The elevated expression of various FGFs has been reported in a diverse range of tumour types such as bladder, renal cell and prostate (amongst others). FGF has also been described as a powerful angiogenic factor. The expression of FGFRs in endothelial cells has also been reported. Activating mutations of various FGFRs have been associated with bladder cancer and multiple myeloma (amongst others) whilst receptor expression has also been documented in prostate and bladder cancer amongst others (Reviewed in Grose, R. et. al., Cytokine & Growth Factor Reviews 2005, 16, p 179-186 and Kwabi-Addo, B. et. al., Endocrine-Related Cancer 2004, 11, p 709-724). For these reasons, the FGF signalling system is an attractive therapeutic target, particularly since therapies targeting FGFRs and/or FGF signalling may affect both the tumour cells directly and tumour angiogenesis.

Pyrazole amides that may find use in therapies targeting FGFRs and/or FGF signalling as FGFR are described PCT Application No. PCT/GB2007/004917 filed on 20 Dec. 2007. In particular, certain pyrazole amides that are described were prepared as racemic mixtures. It has now been found that for certain enatiomers there may be differences in one or more biological and or physiological property which may be advantageous.

In accordance with the present invention, there is provided a compound of formula (Ia) or (Ib):

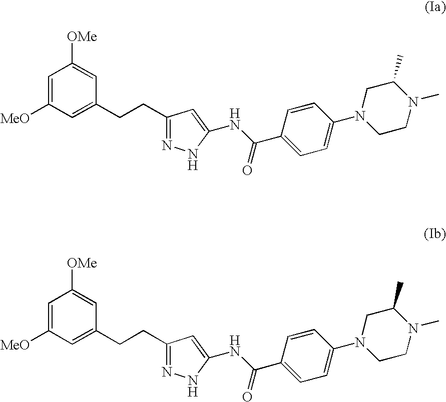

or a pharmaceutically acceptable salt thereof.

In further aspect of the present invention, there is provided a compound of formula (Ib):

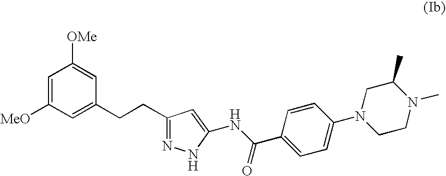

or a pharmaceutically acceptable salt thereof.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The invention relates to any and all tautomeric forms of the compounds of the formula (Ia) and (Ib) that possess FGFR inhibitory activity. For example, the compound of formula (IA) is a tautomer of the compound of formula (Ia).

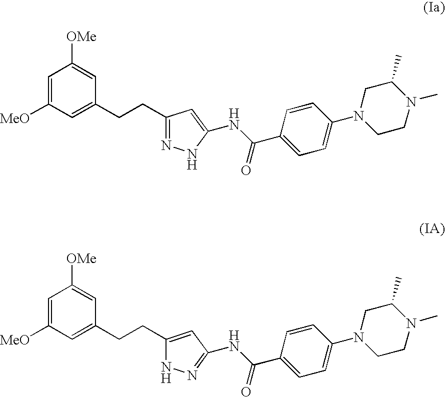

For example, the compound of formula (IB) is a tautomer of the compound of formula (Ib).

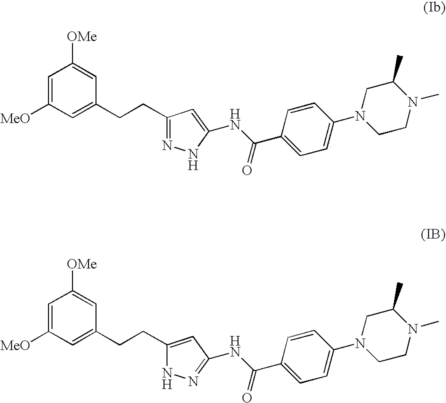

It is also to be understood that certain compounds of the formula (Ia) and (Ib) can exist in solvated as well as unsolvated forms such as, for example, hydrated forms. It is to be understood that the invention encompasses all such solvated forms that possess FGFR inhibitory activity.

In another aspect of the invention, particular compounds of the invention are any one of the Examples or pharmaceutically acceptable salts of any one thereof.

The present invention further provides a process for the preparation of a compound of formula (Ia) or (Ib) as defined herein, or a pharmaceutically acceptable salt thereof, which comprises reacting a compound of formula (IIa) or (IIb)

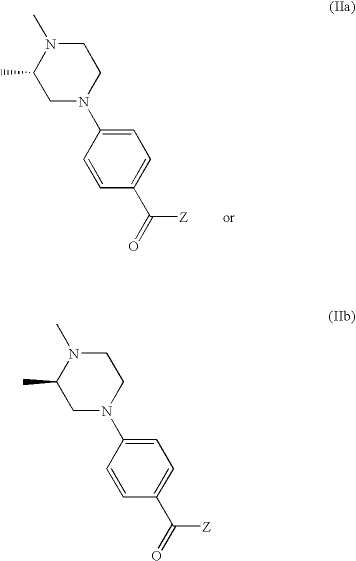

wherein Z represents a leaving group (e.g. halogen, for example chlorine, —CN, —N₃, —OH or a —OR, —OC(O)R, —OCR(NR^{a}R^{b})₂ or —OC(═NR)NR^{a}R^{b} group where R is an optionally substituted alkyl, aryl, heteroaryl or alkaryl and each R^{a}, R^{b} independently is hydrogen or an optionally substituted alkyl, aryl or alkaryl), with a compound of formula (III)

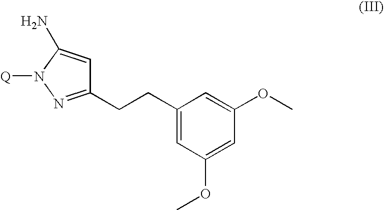

wherein Q is hydrogen or a protecting group (for example t-Bu or BOC group or as described in ‘Protective Groups in Organic Synthesis’, 2nd edition, T. W. Greene and P. G. M. Wuts, Wiley-Interscience (1991)), to give a compound of formual (Ia) or (Ib), and optionally carrying out one or more of the following:
converting the compound obtained to a compound of the invention
forming a pharmaceutically acceptable salt of the compound.

Suitable compounds of Formula (IIa) or (IIb) include carboxylic acids or reactive derivatives of a carboxylic acid. Carboxylic acids or reactive derivatives of a carboxylic acid include acyl halides, such as an acyl chloride formed by the reaction of the acid with an inorganic acid chloride, for example thionyl chloride; a mixed anhydride, for example an anhydride formed by the reaction of the acid with a chloroformate such as isobutyl chloroformate; an active ester, for example an ester formed by the reaction of a carboxylic acid with a phenol such as pentafluorophenol, with an ester, such as pentafluorophenyl trifluoroacetate, or with an alcohol such as methanol, ethanol, isopropanol, butanol or N-hydroxybenzotriazole; an acyl azide, for example an azide formed by the reaction of the acid with an azide such as diphenylphosphoryl azide; an acyl cyanide, for example a cyanide formed by the reaction of an acid with a cyanide such as diethylphosphoryl cyanide; or the product of the reaction of the acid with a carbodiimide such as dicyclohexylcarbodiimide or with a uronium compound such as 2-(7-azabenzotriazol-1-yl)-1,1,3,3-tetramethyluronium hexafluorophosphate(V).

The reaction may conveniently be carried out in the presence of a suitable inert solvent or diluent, for example a halogenated solvent such as methylene chloride, chloroform or carbon tetrachloride, an alcohol or ester such as methanol, ethanol, isopropanol or ethyl acetate, an ether such as tetrahydrofuran or 1,4-dioxan, an aromatic solvent such as toluene. The reaction can also be carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, −20° C. to 100° C., preferably between 0° C. to ambient temperature, dependant upon the reaction being carried out and the nature of the leaving group Z.

The reaction typically can be carried out in the presence of a base. Suitable bases include organic amine bases, such as pyridine, 2,6-lutidine, N,N-diisopropylethylamine, collidine, 4-dimethylaminopyridine, triethylamine, morpholine, N-methylmorpholine or diazabicyclo[5.4.0]undec-7-ene, alkali or alkaline earth metal carbonates or hydroxides, such as sodium carbonate, potassium carbonate, calcium carbonate, sodium hydroxide or potassium hydroxide, alkali metal amides, such as sodium hexamethyldisilazide (NaHMDS), or alkali metal hydrides, such as sodium hydride, dependant upon the reaction being carried out and the nature of the leaving group Z.

The reaction can also be carried out in the presence of a Lewis acid, for example trimethylaluminium, dependant upon the reaction being carried out and the nature of the leaving group Z.

Compounds of formula (IIa), (IIb) or (III) are either commercially available, are known in the literature or may be prepared using known techniques.

Compounds of formula (II), wherein Z is halogen or —OR, may be prepared from compounds of formula (II) wherein Z is —OH by methods known in the literature. For example, methods known for the preparation of acid chlorides or esters from carboxylic acids may be employed.

Compounds of formula (II), wherein Z is —OR, may be prepared by reaction of 2-methylpiperazine with 4-fluorobenzoate esters to give the ester of 4(3-methylpiperazinyl)benzoate, and then followed by N-methylation.

Compounds of formula (III) may be prepared by reacting a compound of formula (IV)

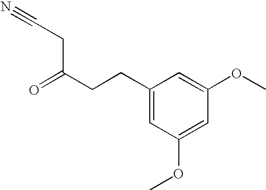

with a hydrazine of formula (V)

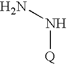

The reaction may be conveniently carried out in a solvent, such as ethanol, at temperature range of 60 to 80° C.

Compounds of formula (IV) and (V) are commercially available compounds, or they are known in the literature, or they are prepared by standard processes known in the art.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (Ia) or (Ib) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley-Interscience (1991).

The compounds of formula (Ia) or (Ib) above may be converted to a pharmaceutically acceptable salt, for example an acid addition salt such as a hydrochloride, hydrobromide, phosphate, acetate, fumarate, maleate, tartrate, citrate, oxalate, methanesulphonate or p-toluenesulphonate, or an alkali metal salt such as a sodium or potassium salt.

The use of tautomers and mixtures thereof also form an aspect of the present invention.

The compound of Formula (1b) may exist in one or more crystalline forms. In a particular aspect of the invention, the compound of Formula (1b) is in a crystalline form, referred to as Form A.

In another aspect the invention there is provided a pharmaceutical composition as defined herein wherein the compound of Formula (1b) is in crystalline form.

In yet another aspect the invention there is provided a pharmaceutical composition as defined herein comprising a compound of Formula (1b) substantially as crystalline Form A.

Substantially as crystalline Form A means that there is greater than 95% of Form A present. In particular there is greater than 96% Form A. Particularly there is greater than 97% Form A. In particular there is greater than 98% Form A. Particularly there is greater than 99% Form A. In particular there is greater than 99.5% Form A. Particularly there is greater than 99.8% Form A.

In one embodiment, there is provided a crystalline form of a compound of Formula (1b) having an XRD pattern comprising peaks at 2-theta (λ=1.5418 Å) 5.288, 17.935, 18.011, 20.513 and 20.753.

In a further embodiment, there is provided a crystalline form of a compound of Formula (1b) having an XRD pattern comprising peaks at 2-theta (λ=1.5418 Å) 5.288, 17.935, 18.011, 18.766, 19.628, 19.667, 20.513, 20.753 and 23.892.

In a further embodiment, there is provided a crystalline form of a compound of Formula (1b) having an XRD pattern comprising peaks at 2-theta (λ=1.5418 Å) 5.288, 17.761, 17.935, 18.011, 18.766, 19.094, 19.628, 19.667, 20.513, 20.753, 22.264 and 23.892.

In a further embodiment, there is provided a crystalline form of a compound of Formula (1b) having an XRD pattern comprising peaks at 2-theta (λ=1.5418 Å) 5.288, 10.483, 11.388, 11.912, 14.086, 15.671, 16.322, 17.761, 17.935, 18.011, 18.766, 19.094, 19.628, 19.667, 20.513, 20.753, 21.765, 22.264, 22.766, 22.793 and 23.892.

In a further embodiment, there is provided a crystalline form of a compound of Formula (1b) having an XRD pattern comprising peaks at 2-theta (λ=1.5418 Å) 3.642, 4.357, 5.288, 7.226, 9.062, 9.482, 10.483, 11.388, 11.85, 11.912, 13.078, 14.086, 14.559, 15.671, 16.156, 16.179, 16.322, 17.761, 17.935, 18.011, 18.766, 19.094, 19.628, 19.667, 20.513, 20.753, 21.765, 22.264, 22.766, 22.793, 23.892, 26.257 and 36.269.

A person skilled in the art will appreciate that the diffraction pattern data presented herein is not to be construed as absolute (for further information see Jenkins, R & Snyder, R. L. ‘Introduction to X-Ray Powder Diffractometry’ John Wiley & Sons, 1996). Therefore, it shall be understood that the crystalline form is not intended to be limited to the crystals that provide X-ray powder diffraction patterns identical to the X-ray powder diffraction patterns described herein. The present invention also includes any crystals providing X-ray powder diffraction patterns substantially the same as those described herein. A person skilled in the art of X-ray powder diffraction is able to judge the substantial similarity of X-ray powder diffraction patterns and will understand that differences may be the result of various factors for example measurement errors resulting from measurement conditions (such as equipment, sample preparation or the machine used); intensity variations resulting from measurement conditions and sample preparation; relative intensity variations of peaks resulting from variations in size or non-unitary aspect ratios of cyrstals; and the position of reflections which can be affected by the precise height at which the sample sits in the diffractometer and the zero calibration of the diffractometer, and surface planarity of the sample.

The compounds of formula (Ia) or (Ib) have activity as pharmaceuticals, in particular as modulators or inhibitors of FGFR activity, and may be used in the treatment of proliferative and hyperproliferative diseases/conditions, examples of which include the following cancers:
(1) carcinoma, including that of the bladder, brain, breast, colon, kidney, liver, lung, ovary, pancreas, prostate, stomach, cervix, colon, thyroid and skin;
(2) hematopoietic tumors of lymphoid lineage, including acute lymphocytic leukaemia, B-cell lymphoma and Burketts lymphoma;
(3) hematopoietic tumours of myeloid lineage, including acute and chronic myelogenous leukaemias and promyelocytic leukaemia;
(4) tumours of mesenchymal origin, including fibrosarcoma and rhabdomyosarcoma; and
(5) other tumours, including melanoma, seminoma, tetratocarcinoma, neuroblastoma and glioma.

In one embodiment the compounds of the invention are useful in the treatment of tumors of the bladder, breast and prostate and multiple myeloma.

Thus, the present invention provides a compound of formula (Ia) or (Ib), or a pharmaceutically-acceptable salt thereof, as herein defined for use in therapy.

According to a further aspect of the present invention there is provided a compound of the formula (Ia) or (Ib), or a pharmaceutically acceptable salt thereof, as defined hereinbefore for use in a method of treatment of the human or animal body by therapy.

In a further aspect, the present invention provides the use of a compound of formula (Ia) or (Ib), or a pharmaceutically acceptable salt, as herein defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating cancer which comprises administering to a patient in need thereof a therapeutically effective amount of a compound of formula (Ia) or (Ib), or a pharmaceutically acceptable salt thereof, as herein defined.

We have found that the compounds defined in the present invention, or a pharmaceutically acceptable salt thereof, are effective anti-cancer agents which property is believed to arise from modulating or inhbiting FGFR activity. Accordingly the compounds of the present invention are expected to be useful in the treatment of diseases or medical conditions mediated alone or in part by FGFR, i.e. the compounds may be used to produce a FGFR inhibitory effect in a warm-blooded animal in need of such treatment.

Thus the compounds of the present invention provide a method for treating cancer characterised by inhibition of FGFR, i.e. the compounds may be used to produce an anti-cancer effect mediated alone or in part by the inhibition of FGFR.

Such a compound of the invention is expected to possess a wide range of anti-cancer properties as activating mutations in FGFR have been observed in many human cancers, including but not limited to breast, bladder, prostrate and multiple myeloma. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In one embodiment compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those tumours which are associated with FGFR, especially those tumours which are significantly dependent on FGFR for their growth and spread, including for example, certain tumours of the bladder, prostrate, breast and multiple myeloma.

Thus according to this aspect of the invention there is provided a compound of the formula (Ia) or (Ib), or a pharmaceutically acceptable salt thereof, as defined herein for use as a medicament.

According to a further aspect of the invention there is provided the use of a compound of the formula (Ia) or (Ib), or a pharmaceutically acceptable salt thereof, as defined herein in the manufacture of a medicament for use in the production of a FGFR inhibitory effect in a warm-blooded animal such as man.

According to this aspect of the invention there is provided the use of a compound of the formula (Ia) or (Ib), or a pharmaceutically acceptable salt thereof, as defined herein in the manufacture of a medicament for use in the production of an anti-cancer effect in a warm-blooded animal such as man.

According to a further feature of the invention, there is provided the use of a compound of the formula (Ia) or (Ib), or a pharmaceutically acceptable salt thereof, as defined herein in the manufacture of a medicament for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (Ia) or (Ib), or a pharmaceutically acceptable salt thereof, as defined herein in the production of a FGFR inhibitory effect in a warm-blooded animal such as man.

According to this aspect of the invention there is provided the use of a compound of the formula (Ia) or (Ib), or a pharmaceutically acceptable salt thereof, as defined herein in the production of an anti-cancer effect in a warm-blooded animal such as man.

According to a further feature of the invention, there is provided the use of a compound of the formula (Ia) or (Ib), or a pharmaceutically acceptable salt thereof, as defined herein in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a FGFR inhibitory effect in a warm-blooded animal, such as man, in need of such treatment which comprises administering to said animal an effective amount of a compound of formula (Ia) or (Ib), or a pharmaceutically acceptable salt thereof, as defined herein.

According to a further feature of this aspect of the invention there is provided a method for producing an anti-cancer effect in a warm-blooded animal, such as man, in need of such treatment which comprises administering to said animal an effective amount of a compound of formula (Ia) or (Ib), or a pharmaceutically acceptable salt thereof, as defined herein.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (Ia) or (Ib) or a pharmaceutically acceptable salt thereof as defined herein.

In a further aspect of the invention there is provided a pharmaceutical composition which comprises a compound of the formula (Ia) or (Ib), or a pharmaceutically acceptable salt thereof, as defined herein in association with a pharmaceutically-acceptable diluent or carrier for use in the production of a FGFR inhibitory effect in a warm-blooded animal such as man.

In a further aspect of the invention there is provided a pharmaceutical composition which comprises a compound of the formula (Ia) or (Ib), or a pharmaceutically acceptable salt thereof, as defined herein in association with a pharmaceutically-acceptable diluent or carrier for use in the production of an anti-cancer effect in a warm-blooded animal such as man.

In a further aspect of the invention there is provided a pharmaceutical composition which comprises a compound of the formula (Ia) or (Ib), or a pharmaceutically acceptable salt thereof, as defined herein in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries in a warm-blooded animal such as man.

The compounds of formula (Ia) or (Ib) and pharmaceutically acceptable salts thereof may be used on their own but will generally be administered in the form of a pharmaceutical composition in which the formula (Ia) or (Ib) compound or salt (active ingredient) is in association with a pharmaceutically acceptable adjuvant, diluent or carrier. Depending on the mode of administration, the pharmaceutical composition may comprise from 0.01 to 99% w (percent by weight), from 0.05 to 80% w, from 0.10 to 70% w, and or even from 0.10 to 50% w, of active ingredient, all percentages by weight being based on total composition.

The present invention also provides a pharmaceutical composition comprising a compound of formula (Ia) or (Ib), or a pharmaceutically acceptable salt thereof, as herein defined, in association with a pharmaceutically acceptable adjuvant, diluent or carrier.

The invention further provides a process for the preparation of a pharmaceutical composition of the invention which comprises mixing a compound of formula (Ia) or (Ib), or a pharmaceutically acceptable salt thereof, as herein defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The size of the dose for therapeutic purposes of a compound of the invention will naturally vary according to the nature and severity of the conditions, the age and sex of the animal or patient and the route of administration, according to well known principles of medicine.

In general, a compound of the invention will be administered so that a daily dose in the range, for example, from 0.1 mg to 1000 mg active ingredient per kg body weight is received, given if required in divided doses. However the daily dose will necessarily be varied depending upon the host treated, the particular route of administration, and the severity of the illness being treated. Accordingly the optimum dosage may be determined by the practitioner who is treating any particular patient. In general lower doses will be administered when a parenteral route is employed. Thus, for example, for intravenous administration, a dose in the range, for example, from 0.1 mg to 30 mg active ingredient per kg body weight will generally be used. Similarly, for administration by inhalation, a dose in the range, for example, from 0.1 mg to 25 mg active ingredient per kg body weight will generally be used. Oral administration is however preferred. For example, a formulation intended for oral administration to humans will generally contain, for example, from 0.1 mg to 2 g of active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of

5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as famesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD 1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669,

WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

### EXAMPLES

The invention will now be further described with reference to the following illustrative examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz, in DMSO-d₆ unless otherwise indicated;
(viii) chemical symbols have their usual meanings; SI units and symbols are used;
(ix) solvent ratios are given in volume:volume (v/v) terms; and
(x) mass spectra (MS) data was generated on an LC/MS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)+ for positive ion mode and (M−H)− for negative ion mode;
(xi) Preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88) and acetonitrile;
(xii) the following abbreviations have been used:
   THF tetrahydrofuran;
   DMF N,N-dimethylformamide;
   EtOAc ethyl acetate;
   DCM dichloromethane; and
   DMSO dimethylsulphoxide
   DIPEA N,N-diisopropylethylamine
      (also known as N-ethyl-N-propan-2-yl-propan-2-amine)
   PBS phosphate buffered saline
   HEPES N-[2-Hydroxyethyl]piperazine-N′-[2-ethanesulfonic acid]
   DTT dithiothreitol
   ATP Adenosine Triphosphate
   BSA bovine serum albumin
   DMEM Dulbecco's modified Eagle's Medium
   MOPS 3-(N-morpholino)propanesulfonic acid
(xiii) compounds are named using proprietary naming software: Openeye Lexichem version 1.4, using IUPAC naming convention;
(xiv) unless otherwise specified, starting materials are commercially available.

### Example 1(a)

### Preparation of (S)—N-(3-(3,5-Dimethoxyphenethyl)-1H-pyrazol-5-yl)-4-(3,4-dimethylpiperazin-1-yl)benzamide

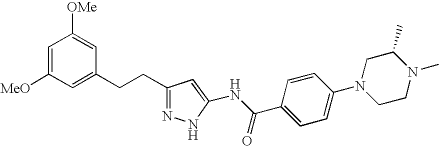

Trimethylaluminum (2M in toluene, 1.93 ml, 3.87 mmol) was added dropwise to a suspension of 3-(3,5-dimethoxyphenethyl)-1H-pyrazol-5-amine (382 mg, 1.55 mmol) and (S)-methyl 4-(3,4-dimethylpiperazin-1-yl)benzoate (384 mg, 1.55 mmol) in toluene (10 ml) at 25° C. The resulting solution was stirred at 60° C. for 18 hours. The reaction mixture was poured into methanol (50 ml) and acidified with 2M hydrochloric acid. The crude product was purified by ion exchange chromatography, using an SCX column. The desired product was eluted from the column using 7M methanolic ammonia and evaporated to dryness to afford impure product. The crude product was purified by preparative HPLC (Waters XTerra C18 column, 5μ silica, 30 mm diameter, 100 mm length), using decreasingly polar mixtures of 1% aqueous ammonia and acetonitrile as eluent. Fractions containing the desired compound were evaporated to dryness to give (S)—N-(3-(3,5-dimethoxyphenethyl)-1H-pyrazol-5-yl)-4-(3,4-dimethylpiperazin-1-yl)benzamide (387 mg, 54%) as a white solid.

¹H NMR (399.9 MHz, DMSO-d6) δ 1.07 (3H, d), 2.09-2.14 (1H, m), 2.18-2.25 (1H, m), 2.22 (3H, s), 2.44-2.53 (1H, m), 2.77-2.87 (2H, m), 2.88 (4H, s), 3.65-3.75 (2H, m), 3.73 (6H, s), 6.34 (1H, t), 6.43 (2H, d), 6.46 (1H, s), 6.96 (2H, d), 7.91 (2H, d), 10.31 (1H, s), 12.09 (1H, s). MS: m/z 464 (MH+).

### (S)-Methyl 4-(3,4-dimethylpiperazin-1-yl)benzoate

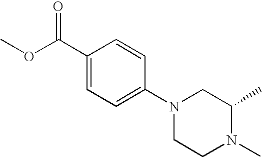

Sodium triacetoxyborohydride (2.25 g, 10.6 mmol) was added to (S)-methyl 4-(3-methylpiperazin-1-yl)benzoate (0.99 g, 4.24 mmol), formaldehyde (6.32 ml of a 37% aqueous solution, 84.9 mmol) and acetic acid (0.49 ml, 8.49 mmol) in methanol (21 ml) at 25° C. The resulting solution was stirred at ambient temperature under nitrogen for 18 hours. The reaction mixture was basified with saturated aqueous NaHCO₃ solution and concentrated under reduced pressure. The residue was diluted with EtOAc (200 ml), and washed sequentially with saturated aqueous NaHCO₃ solution (200 ml). The aqueous was further extracted with EtOAc (2×75 ml) and the combined organics washed with water (200 ml) and saturated brine (200 ml). The organic layer was dried over MgSO₄, filtered and evaporated to afford crude product. The crude product was purified by flash silica chromatography, eluting with EtOAc/isohexane (0:100 increasing in polarity to 100:0). Pure fractions were evaporated to dryness to give (S)-methyl 4-(3,4-dimethylpiperazin-1-yl)benzoate (0.538 g, 51%) as a white solid.

¹H NMR (399.9 MHz, CDCl₃) δ 1.14-1.15 (3H, m), 2.18-2.26 (1H, m), 2.34 (3H, s), 2.35-2.41 (1H, m), 2.61-2.67 (1H, m), 2.87-2.91 (1H, m), 2.99-3.06 (1H, m), 3.58-3.62 (1H, m), 3.66-3.70 (1H, m), 3.86 (3H, s), 6.84-6.87 (2H, m), 7.89-7.93 (2H, m). MS: m/z 249 (MH+).

### (S)-Methyl 4-(3-methylpiperazin-1-yl)benzoate

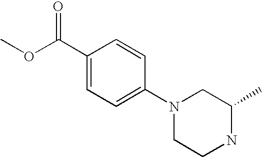

Methyl 4-fluorobenzoate (1.00 ml, 7.73 mmol) was added to (S)-(+)-2-methylpiperazine (1.55 g, 15.5 mmol) in DMA (31 ml) at 25° C. The resulting solution was stirred under nitrogen at 120° C. for 3 days. The reaction mixture was concentrated and diluted with EtOAc (100 ml), and washed sequentially with saturated aqueous NaHCO₃ solution (200 ml). The aqueous was further extracted with EtOAc (2×100 ml), and the combined organics washed with water (200 ml) and saturated brine (200 ml). The organic layer was dried over MgSO₄, filtered and evaporated to give desired (S)-methyl 4-(3-methylpiperazin-1-yl)benzoate (0.995 g, 55%) as a brown oil which crystallized on standing. This was used directly with no further purification.

¹H NMR (399.9 MHz, CDCl₃) δ 1.14-1.15 (3H, d), 2.44-2.50 (1H, m), 2.80-2.86 (1H, m), 2.91-3.03 (2H, m), 3.10-3.15 (1H, m), 3.67-3.70 (2H, m), 3.86 (3H, s), 6.85-6.87 (2H, m), 7.89-7.93 (2H, m), NH not observed. MS: m/z 235 (MH+).

### 3-(3,5-dimethoxyphenethyl)-1H-pyrazol-5-amine

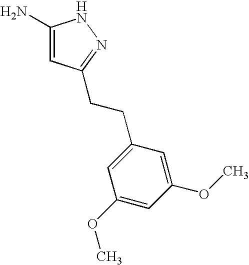

used as starting material was prepared as follows:

Acetonitrile (2.29 ml, 43.61 mmol, 1.2 eq) was added to a slurry of sodium hydride (1.75 g dispersion in mineral oil, 43.61 mmol, 1.2 eq) in anhydrous toluene (70 ml) and the mixture stirred at room temperature for 30 mins. Ethyl 3-(3,5-dimethoxyphenyl)propanoate (8.66 g, 36.34 mmol, 1 eq) in toluene (60 ml) was added and the reaction was refluxed for 18 h. After cooling, the reaction mixture was quenched with water and the solvent was evaporated under reduced pressure. The residue was dissolved in 2M HCl (50 ml). The acidic solution was extracted with ethyl acetate. The organic extracts were combined and washed with water, brine and dried over magnesium sulphate. After filtering, the solvent was evaporated under reduced pressure to yield the crude product as a yellow oil. The oil was purified by silica column chromatography (eluting with DCM) and the desired fractions were combined and evaporated to yield a cream solid (3.76 g, 44% yield).

To the cream solid (3.72 g, 15.96 mmol, 1 eq) in ethanol (55 ml) was added hydrazine hydrate (852 μl, 17.56 mmol, 1.1 eq). The reaction was refluxed for 24 h before allowing to cool. After evaporation under reduced pressure, the residue was extracted into DCM. The organic layers were washed with water, brine, dried with magnesium sulphate, filtered and evaporated under reduced pressure to afford 3-(3,5-dimethoxyphenethyl)-1H-pyrazol-5-amine as a pale yellow solid (3.76 g. 42% over 2 steps).

¹H NMR (300.132 MHz, DMSO) δ 2.64-2.82 (4H, m), 3.71 (6H, s), 4.07-4.72 (2H, m), 5.20 (1H, s), 6.31 (1H, t), 6.38 (2H, d). MS: m/z 248 (MH+)

### Example 1(b)(i)

### Preparation of (R)—N-(3-(3,5-Dimethoxyphenethyl)-1H-pyrazol-5-yl)-4-(3,4-dimethylpiperazin-1-yl)benzamide

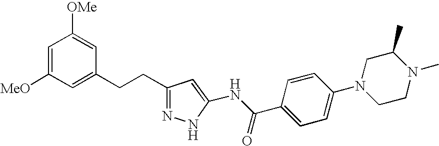

Trimethylaluminum (2M in toluene, 2.44 ml, 4.88 mmol) was added dropwise to a suspension of 3-(3,5-dimethoxyphenethyl)-1H-pyrazol-5-amine (483 mg, 1.95 mmol) and (R)-methyl 4-(3,4-dimethylpiperazin-1-yl)benzoate (485 mg, 1.95 mmol) in toluene (10 ml) at 25° C. The resulting solution was stirred at 60° C. for 18 hours. The reaction mixture was poured into methanol (50 ml) and acidified with 2M hydrochloric acid. The crude product was purified by ion exchange chromatography, using an SCX column. The desired product was eluted from the column using 7M methanolic ammonia and evaporated to dryness to afford impure product. The crude product was purified by preparative HPLC (Waters XTerra C18 column, 5μ silica, 30 mm diameter, 100 mm length), using decreasingly polar mixtures of 1% aqueous ammonia and acetonitrile as eluent. Fractions containing the desired compound were evaporated to dryness to give (R)—N-(3-(3,5-dimethoxyphenethyl)-1H-pyrazol-5-yl)-4-(3,4-dimethylpiperazin-1-yl)benzamide (561 mg, 62%) as a white solid.

¹H NMR (399.9 MHz, DMSO-d6) δ 1.07 (3H, d), 2.09-2.14 (1H, m), 2.18-2.25 (1H, m), 2.22 (3H, s), 2.44-2.53 (1H, m), 2.77-2.87 (2H, m), 2.88 (4H, s), 3.65-3.75 (2H, m), 3.73 (6H, s), 6.34 (1H, t), 6.43 (2H, d), 6.46 (1H, s), 6.96 (2H, d), 7.91 (2H, d), 10.31 (1H, s), 12.09 (1H, s). MS: m/z 464 (MH+).

### (R)-Methyl 4-(3,4-dimethylpiperazin-1-yl)benzoate

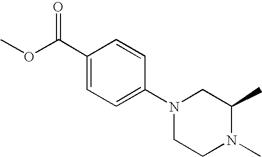

Sodium triacetoxyborohydride (1.62 g, 7.63 mmol) was added to (R)-methyl 4-(3-methylpiperazin-1-yl)benzoate (0.715 g, 3.05 mmol), formaldehyde (4.54 ml of a 37% aqueous solution, 61 mmol) and acetic acid (0.35 ml, 6.10 mmol) in methanol (15 ml) at 25° C. The resulting solution was stirred at ambient temperature under nitrogen for 18 hours. The reaction mixture was basified with saturated aqueous NaHCO₃ solution and concentrated under reduced pressure. The reaction mixture was diluted with EtOAc (200 ml), and washed sequentially with saturated aqueous NaHCO₃ solution (200 ml). The aqueous was further extracted with EtOAc (2×75 ml) and the combined organics washed with water (200 ml) and saturated brine (200 ml). The organic layer was dried over MgSO₄, filtered and evaporated to afford crude product. The crude product was purified by flash silica chromatography, eluting with EtOAc/isohexane (30:70 increasing in polarity to 100:0). Pure fractions were evaporated to dryness to give (R)-methyl 4-(3,4-dimethylpiperazin-1-yl)benzoate (0.600 g, 79%) as a cream solid.

¹H NMR (399.9 MHz, CDCl₃) δ 1.14-1.15 (3H, d), 2.19-2.24 (1H, m), 2.34 (3H, s), 2.35-2.41 (1H, m), 2.61-2.67 (1H, m), 2.87-2.91 (1H, m), 2.99-3.06 (1H, m), 3.58-3.62 (1H, m), 3.66-3.71 (1H, m), 3.86 (3H, s), 6.84-6.87 (2H, m), 7.89-7.93 (2H, m). MS: m/z 249 (MH+).

### (R)-Methyl 4-(3-methylpiperazin-1-yl)benzoate

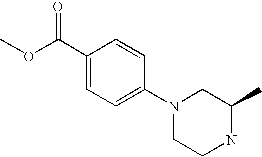

Methyl 4-fluorobenzoate (0.62 ml, 4.79 mmol) was added to (R)-(−)-2-methylpiperazine (0.96 g, 9.58 mmol) in DMA (19 ml) at 25° C. The resulting solution was stirred under nitrogen at 120° C. for 50 hours. The reaction mixture was concentrated and diluted with EtOAc (100 ml), and washed sequentially with saturated aqueous NaHCO₃ solution (200 ml). The aqueous was further extracted with EtOAc (2×100 ml) and the combined organics washed with water (200 ml) and saturated brine (200 ml). The organic layer was dried over MgSO₄, filtered and evaporated to give (R)-methyl 4-(3-methylpiperazin-1-yl)benzoate (0.721 g, 64%) as a brown solid. This was used directly with no further purification.

¹H NMR (399.9 MHz, CDCl₃) δ 1.14-1.16 (3H, m), 2.45-2.51 (1H, m), 2.80-2.87 (1H, m), 2.93-3.04 (2H, m), 3.11-3.15 (1H, m), 3.66-3.71 (2H, m), 3.86 (3H, s), 6.84-6.88 (2H, m), 7.89-7.93 (2H, m), NH not observed. MS: m/z 235 (MH+).

### Example 1(b)(ii)

### Alternate Preparation of (R)—N-(3-(3,5-Dimethoxyphenethyl)-1H-pyrazol-5-yl)-4-(3,4-dimethylpiperazin-1-yl)benzamide

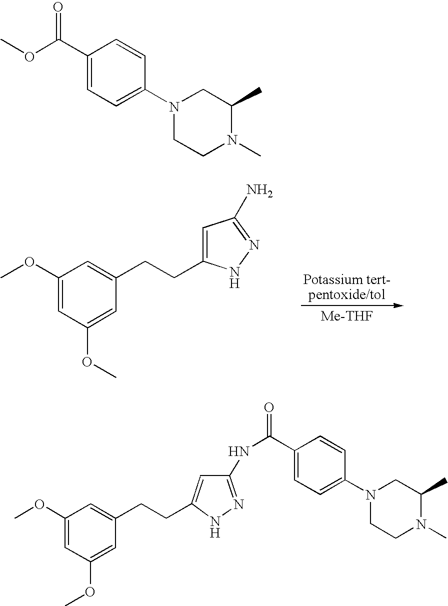

A solution of 5-(3,5-dimethoxyphenethyl)-1H-pyrazole-3-amine (159 g, 0.643 mol, 1 eq) and (R)-Methyl 4-(3,4-dimethylpiperazin-1-yl)benzoate (191 g, 0.772 mol, 1.2 eq) in 2-methyltetrahydrofuran (3500 ml) was heated to reflux using Dean-Stark apparatus. Approximately 300 ml of solvent was removed and the solution was allowed to cool to 70° C. A solution of potassium tert-pentoxide in toluene (25%, 1.7M, 908 ml, 1.544 mol, 2.4 eq) was added over 30 min. The resultant suspension was heated to reflux and a total of 1.5L of solvent was removed under Dean-Stark conditions over 75 min. The suspension was allowed to cool to 40° C. and LC showed complete conversion.

Water (20 ml) and 2M hydrochloric acid (380 ml, 0.76 mol) were added carefully (exotherm from 40° C. to 50° C.). A dark solution formed and this was diluted with isopropyl acetate (1500 ml). The aqueous layer was separated and the organic layer was washed with water (500 ml) and brine (500 ml). The organic layer was dried over magnesium sulphate and evaporated in vacuo at 45° C. to leave a dark gum. Isopropyl acetate (960 ml) was added and the mixture rotated at 55° C. for 60 min. The gum slowly dissolved and a white solid precipitated. The slurry was cooled to 0° C. and stirred for 120 min. The product was collected by filtration, washed with cold isopropyl acetate (2×400 ml) and dried at 50° C. for 120 min. The product was obtained as a white solid (231 g, 78%). ¹H NMR (CDCl₃) was consistent with the data from Example 1(b)(i). LC: 99.5% purity.

### (R)-Methyl 4-(3,4-dimethylpiperazin-1-yl)benzoate

(R)-Methyl 4-(3-methylpiperazin-1-yl)benzoate (218 g, 0.93 mol, 1 eq) was dissolved in methanol (2200 ml, 10 vols) and acetic acid (111.7 g, 1.86 mol, 2 eq). 37% aqueous formaldehyde solution (880 ml, 11.74 mol, 12.6 eq) was added and a solid precipitated. The mixture was stirred whilst sodium triacetoxyborohydride (493 g, 2.33 mol, 2.5 eq) was added portionwise over 1 hour. The temperature was kept below 30° C. The resultant solution was stirred under nitrogen overnight.

Methanol was removed in vacuo at 45° C. and the residue was poured carefully into a saturated solution of sodium hydrogen carbonate (5000 ml). The mixture was stirred for 15 minutes under a stream of nitrogen and a solid precipitated. The product was extracted with isopropyl acetate (2000 ml and 1500 ml). The combined organic layers were washed with saturated sodium hydrogen carbonate solution (1000 ml) and dried over magnesium sulphate. The isopropyl acetate solution was concentrated in vacuo and the product started to crystallise at low volumes. The product was collected by filtration and a second crop was obtained (196 g, 85%). ¹H NMR (CDCl₃) was consistent with the data from Example 1(b)(i). LC: 99.5% purity.

### (R)-Methyl 4-(3-methylpiperazin-1-yl)benzoate

A suspension of (R)-2-methylpiperazine (203 g, 2.03 mol, 1.59 eq) in DMSO (1200 ml, 6 vols) was stirred at 37° C. Potassium carbonate (383 g, 2.77 mol, 2.16 eq) was added and the suspension stirred whilst a solution of methyl 4-fluorobenzoate (198 g, 1.28 mol, 1 eq) in DMSO (200 ml, 1 vol) was added over 20 minutes. The reaction mixture was stirred at 95° C. for 24 hours. An IPC showed that less than 1% of methyl-4-fluorobenzoate remained.

The reaction mixture was cooled to 40° C., diluted with ethyl acetate (1600 ml), and poured slowly into water (5000 ml). The mixture was stirred for 5 min and the layers were allowed to separate. The aqueous layer was extracted with ethyl acetate (1600 ml). The combined organic layers were washed with water (1200 ml) and brine (1200 ml). The solvent was removed in vacuo to afford a white solid 233 g (78%). ¹H NMR (CDCl₃) was consistent with the data from Example 1(b)(i). LC: 98.4% purity.

XRD—Form A

Powder X-ray diffraction patterns were recorded using a Bruker D4 X-ray diffractometer (wavelength of X-rays 1.5418 Å Cu source, Voltage 40 kV, filament emission 40 mA). Samples were scanned from 2-40° 2θ using a 0.00570° step and a 0.03 second per step time count.

Peaks were observed at:

| Angle/°2θ (λ = 1.5418 Å) | Relative Intensity/% |
|---|---|
| 36.269 | 6.9 |
| 26.257 | 16.3 |
| 23.892 | 44.9 |
| 22.793 | 24 |
| 22.766 | 22.5 |
| 22.264 | 35.2 |
| 21.765 | 28.3 |
| 20.753 | 49.1 |
| 20.513 | 65.2 |
| 19.667 | 41.7 |
| 19.628 | 40.5 |
| 19.094 | 30.9 |
| 18.766 | 47.5 |
| 18.011 | 82 |
| 17.935 | 60.7 |
| 17.761 | 38.7 |
| 16.322 | 27.2 |
| 16.179 | 15.9 |
| 16.156 | 15 |
| 15.671 | 29 |
| 14.559 | 14.2 |
| 14.086 | 24 |
| 13.078 | 18.4 |
| 11.912 | 20.3 |
| 11.85 | 19.8 |
| 11.388 | 22.5 |
| 10.483 | 20.4 |
| 9.482 | 15.6 |
| 9.062 | 17.6 |
| 7.226 | 13.2 |
| 5.288 | 100 |
| 4.357 | 10.7 |
| 3.642 | 15 |

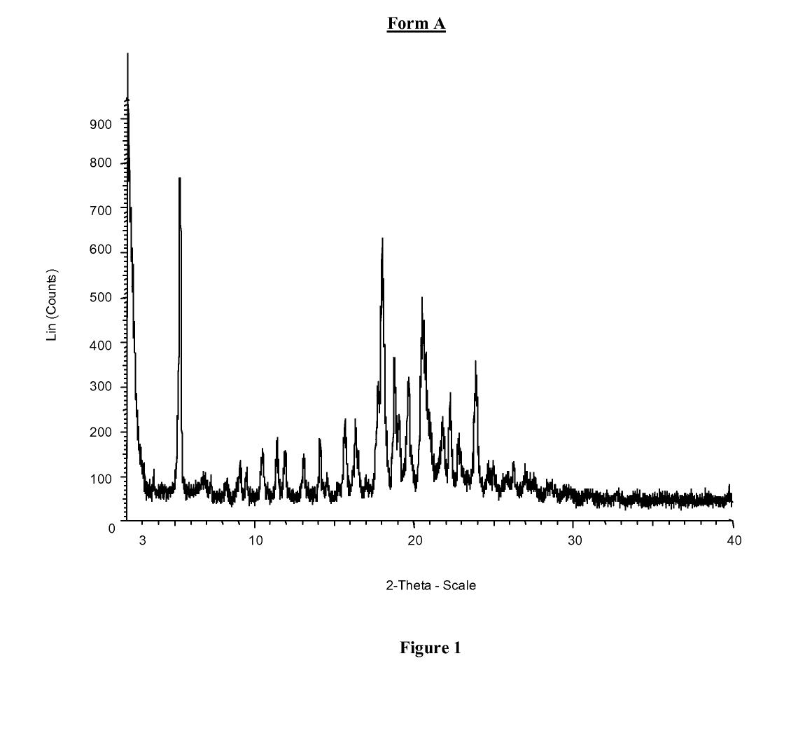

XRD Pattern for Form A is shown in FIG. 1.

### Enzyme Assay

### FGFR1 Kinase Assay—Caliper Echo Dosing

To determine inhibition of FGFR1 activity, kinase assays were conducted using Caliper technology.

Kinase activity assays were performed in Greiner 384-well low volume plates, with a total reaction volume of 12 ul per well. Final concentration of FGFR1 active kinase in each reaction well was 7.2 nM. The substrate for each assay was a custom peptide with fluorescent tag (13 amino acids in length, KKSRGDYMTMQIG with the fluorescene tag on the first K).

Compounds were dispensed directly in to assay plates using a Labcyte Echo 550 acoustic droplet ejection unit. Each well received 120 nl of DMSO containing compound such that the final concentration of compound in the assay prior to the addition of the stop solution ranged between 30 uM and 30 pM. In addition to compounds each plate carried maximum and minimum control wells, the max wells contained 120 nl of DMSO and the min wells contained 120 nl of 10 mM staurosporine (LC Laboratories, MA 01801, USA Catalogue No. S-9300). The Enzyme (at 7.2 nM [final]) and Substrate (at 3.6 uM [final]) were added separately to the compound plates, in reaction buffer [comprising: 50 mM MOPS (Sigma, Catalogue No. M1254)—pH 6.5, 0.004% Triton (Sigma, Catalogue No. X-100), 2.4 mM DTT, 12 mM MgCl₂, 408 uM ATP] resulting in a final DMSO concentration in the reaction mix of 1%.

Assay plates were incubated at room temperature for 1.5 h, before the reaction was stopped with the addition of buffer [comprising: 100 mM HEPES—pH 7.5, 0.033% Brij-35 (Sigma Catalogue No. B4184), 0.22% Caliper Coating Reagent #3 (Caliper Life Sciences Catalogue No. 760050), 88 mM EDTA, 5% DMSO]. Stopped assay plates were then read using the Caliper LabChip® LC3000 (which uses microfludics to measure a shift in mobility between fluorescent labelled peptide and the FGFR1 kinase—phosphorylated form of this peptide).

In the assay, compounds were tested at a range of concentrations. The mean data values for each concentration, along with untreated control wells and 100% inhibition control wells were used to derive a plot of inhibition against concentration. From this data, the IC50 value or a percentage inhibition value at fixed concentration may be determined. Percentage inhibition at 1 uM, as expressed herein, is a calculated value based on the curve fit that was generated experimentally. From the fitted curve plot, the effect of compound at a concentration of 1 uM was calculated as a percentage inhibition. The IC₅₀ is the concentration of compound, which inhibits FGFR1 kinase activity by 50% in the context of this assay. This value is calculated using a standard curve fitting software package Origin™. Where compounds have been tested on more than one occasion the IC₅₀ value may be sited as a geometric mean.

Example 1a—IC₅₀ 0.00074 μM
Example 1b—IC₅₀ 0.0011 μM, 0.00064 μM, 0.00076 μM.

### FGFR4 Kinase Assay—Caliper

To determine inhibition of FGFR4 activity, kinase assays were conducted using Caliper technology. FGFR4 enzyme was used (8 μM, Cat. No. PR4380B, Invitrogen). Kinase activity assays were performed in Greiner 384-well low volume plates, with a total reaction volume of 12 μL per well. The final concentration of FGFR4 active kinase in each reaction well was 25 nM. The substrate for each assay was a custom peptide with fluorescent tag (13 amino acids in length, 5FAM-EEPLYWSFPAKKK—CONH₂) the sequence of which was specific for FGFR4 kinase.

Compounds were serially diluted in 5% (v/v) DMSO, before being added to assay plates. The Enzyme (at 25 nM (final)) and Substrate (at 1.5 μM (final)) were added separately to the compound plates, in reaction buffer (comprising: 100 mM HEPES—pH 7.5, 0.004% Triton, 1 mM DTT (final), 10 mM MnCl₂ (final), 30 μM ATP (final)) resulting in a final DMSO concentration in the reaction mix of 0.8%.

Assay plates were incubated at RT for 2 hours, before the reaction was stopped with the addition of buffer (comprising: 100 mM HEPES—pH7.5, 0.033% Brij-35, 0.22% Caliper Coating Reagent #3, 40 mM EDTA, 5% DMSO). Stopped assay plates were then read using the Caliper LabChip™ LC3000 (which uses microfludics to measure a shift in mobility between fluorescent labelled peptide and the FGFR4 kinase—phosphorylated form of this peptide).

In the assay, compounds were tested at a range of concentrations. The mean data values for each concentration, along with untreated control wells and 100% inhibition control wells were used to derive a plot of inhibition against concentration. From this data, the IC₅₀ value or a percentage inhibition value at fixed concentration may be determined.

Example 1b—IC₅₀ 0.022 μM, 0.016 μM, 0.016 μM.

### Cell Assay

Cell FGFR1 (ECHO)—Cell Based Inhibition of Transiently Expressed FGFR1 IIIc Phosphorylation via use of ECHO Technology (Measured using Phospho-Specific Primary and Fluorescent Secondary Antibodies).

This assay is designed to detect inhibitors of transiently expressed FGFR1 phosphorylation by antibody staining of fixed cells detected using ArrayScan technology.

Cos-1 cells were routinely passaged in DMEM (Gibco BRL, 41966) plus 3% foetal calf serum (FCS), 1% L-glutamine (Gibco BRL, 25030) to a confluence of 80%. To undertake the assay, Cos-1 cells were harvested at 90-95% confluence for cell transfection. For each 96-well plate, 24 μl Lipofectamine 2000 was added to 809 ul OptiMEM and incubated at room temperature for 5 minutes. For each 96 well plate, 20 ug 3′ FLAG tagged FGFR1/pcDNA3.1 (In-house clone15, MSD 4793) was diluted with OptiMEM to a total volume of 833 μl. Equal volumes of DNA and Lipofectamine 2000 were combined (DNA: Lipid=1:1.2 ratio) and incubated at room temperature for 20 minutes.

The harvested Cos-1 cells are counted using a coulter counter and diluted further with 1% FCS/DMEM to 2.5×10⁵ cells/ml. For each 96-well, 8.33 ml cells were required. The complexed transfection solution was added to the cell solution and the cells were seeded at 2.5×10⁵ cells/well in DMEM plus 1% foetal calf serum, 1% L-glutamine in 96 well plates (Costar, 3904) and incubated at 37° C. (+5% CO₂) in a humidified incubator overnight (24 hrs).

The following day, compounds from dry weight samples were dissolved in 100% DMSO to give 10 mM concentration. 40 μl of the compound was dispensed into the wells of each quadrant across the 384 Labcyte plate (Labcyte Catalogue No. P-05525) (inclusive of a positive control (100% DMSO), a negative control (10 μM) and a reference compound (250 nM)). The 384 Labcyte plate was then transferred to the Hydra to dilute the compounds 1:100 into the remaining wells of the quadrant. 70 μl of media was aspirated from the assay plate using the Quadra before the plate was transferred onto the ECHO 550. The 384 Labcyte compound plate was also transferred onto the ECHO 550. Compound transfer to the assay plate on the ECHO 550 was at concentration ranges 1) 10 μM, 2) 3 μM, 3) 1 μM, 4) 0.3 μM, 5) 0.1 μM, 6) 0.01.

The plates were gently tapped to mix compound in with the cell media and left to incubate at 37° C. with 5% CO₂ for 1 hour.

Media was removed from the wells using vacuum aspiration; cells were fixed by adding 50 μl of 100% methanol to each well and incubated at room temperature for 20 minutes. The fixative solution was then removed and the wells were washed once with 200 μl phosphate buffered saline (PBS/A) before permeabilising the cells by the addition of 50 ul/well 0.1% triton/PBS/A for 20 minutes at room temperature. The permeabilisation solution was then removed and the cells washed once more with 200 μl/well PBS/A before the addition of 40 μl 1/1000 primary antibody solution (Cell Signalling Technologies #CS3476; mouse anti-phospho FGFR1 diluted in PBS/A with 10% FCS+0.1% Tween20) to each well. Following incubation at room temperature for 1 hour, the antibody solution was removed and the wells were washed once with 200 ul/well PBS/A. Then 40 μl 1/500 secondary antibody (A11005; goat anti-mouse 594) solution and 1/10000 Hoechst (diluted together in PBS/A with 10% FCS+0.1% Tween 20) were added and the plate incubated in the dark at room temperature for one hour. Finally, the plates were washed once with 200 μl/well PBS/A, leaving the final wash in the wells before sealing the plates. The plates were read on an Arrayscan (Cellomics). The Channel 2 (594 nm) values obtained from undosed (max) and reference compound (min) wells within a plate are used to set boundaries for 0% and 100% compound inhibition. Compound data was normalized against these values to determine the dilution range of a test compound that gives 50% inhibition of phosphorylated FGFR1.

Example 1b—IC₅₀<0.01 μM, 0.0011 μM, 0.0022 μM, 0.0048 μM.

### Cell FGFR2 (ECHO) Assay

Cell based inhibition of constitutively expressed FGFR2 phosphorylation via use of ECHO technology (measured using phospho-specific primary and fluorescent secondary antibodies).

This assay may be used to detect inhibitors of constitutively expressed FGFR2 phosphorylation by antibody staining of fixed cells detected using ArrayScan technology. SUM52-PE cells were routinely passaged in RPMI 1640 (Gibco BRL, 31870) plus 10% foetal calf serum (FCS), 1% L-glutamine (Gibco BRL, 25030) to a confluence of 70%. The harvested SUM52-PE cells are counted using a coulter counter and diluted further with 1% FCS/RPMI 1640 to 1.5×10⁵ cells/mL. For each 96-well plate, 10 mL cells were required. 100 μL of cell suspension was added to each well of 96 well plates (Costar, 3904) and incubated at 37° C. (+5% CO₂) in a humidified incubator overnight (24 hours). The following day, compounds from dry weight samples were dissolved in 100% DMSO to give 100 μM concentration. 40 μL of the compound solution was dispensed into the wells of each quadrant across the 384 Labcyte plate (Labcyte Catalogue No. P-05525) (inclusive of a positive control (100% DMSO), a negative control (10 μM) and a reference compound (250 nM)—1-tert-butyl-3-[2-{[3-(diethylamino)propyl]amino}-6-(3,5-dimethoxyphenyl)-pyrido[2,3-d]pyrimidin-7-yl]urea—PD173074—a commercially available FGFR inhibitor). The 384 Labcyte plate was then transferred to the Hydra to dilute the compounds 1:100 into the remaining wells of the quadrant. 70 μL of media was aspirated from the assay plate using the Quadra before the plate was transferred onto the ECHO 550. The 384 Labcyte compound plate was also transferred onto the ECHO 550. Compound transfer to the assay plate on the ECHO 550 was at concentration ranges (1) 100 nM, (2) 33.3 nM, (3) 11.1 nM, (4) 3.70 nM, (5) 1.23 nM, (6) 0.41 nM. The plates were gently tapped to mix compound in with the cell media and left to incubate at 37° C. with 5% CO₂ for 1 hour. Media was removed from the wells using vacuum aspiration; cells were fixed by adding 50 μL of 100% methanol to each well and incubated at RT for 20 minutes. The fixative solution was then removed and the wells were washed once with 200 μL phosphate buffered saline (PBS/A) before permeabilising the cells by the addition of 50 μL/well 0.1% triton/PBS/A for 20 minutes at RT. The permeabilisation solution was then removed and the cells washed once more with 200 μL/well PBS/A before the addition of 40 μL 1/1000 primary antibody solution (Cell Signalling Technologies #CS3476; mouse anti-phospho FGFR diluted in PBS/A with 10% FCS+0.1% Tween20) to each well. Following incubation at RT for 1 hour, the antibody solution was removed and the wells were washed once with 200 μL/well PBS/A. Then 40 μL 1/500 secondary antibody (A11005; goat anti-mouse 594) solution and 1/10000 Hoechst (diluted together in PBS/A with 10% FCS+0.1% Tween 20) were added and the plate incubated in the dark at RT for 1 hour. Finally, the plates were washed once with 200 μL/well PBS/A, leaving the final wash in the wells before sealing the plates. The plates were read on an Arrayscan (Cellomics). The Channel 2 (594 nm) values obtained from undosed (max) and reference compound (min) wells within a plate are used to set boundaries for 0% and 100% compound inhibition.

### Cell FGFR3 (ECHO) Assay

Cell based inhibition of transiently expressed FGFR3 IIIc phosphorylation via use of ECHO technology (measured using phospho-specific primary and fluorescent secondary antibodies).

This assay may be used to detect inhibitors of transiently expressed FGFR3 phosphorylation by antibody staining of fixed cells detected using ArrayScan technology. Cos-1 cells were routinely passaged in DMEM (Gibco BRL, 41966) plus 3% foetal calf serum (FCS), 1% L-glutamine (Gibco BRL, 25030) to a confluence of 80%. To undertake the assay, Cos-1 cells were harvested at 90-95% confluence for cell transfection. For each 96-well plate, 24 μL Lipofectamine 2000 was added to 809 μL OptiMEM and incubated at RT for 5 minutes. For each 96 well plate, 20 μg 3′ FLAG tagged FGFR3/pcDNA3.2 full-length FGFR3 was diluted with OptiMEM to a total volume of 833 μL. Equal volumes of DNA and Lipofectamine 2000 were combined (DNA: Lipid=1:1.2 ratio) and incubated at RT for 20 minutes. The harvested Cos-1 cells are counted using a coulter counter and diluted further with 1% FCS/DMEM to 2.5×10⁵ cells/mL. For each 96-well, 8.33 mL cells were required. The complexed transfection solution was added to the cell solution and the cells were seeded at 2.1×10⁴ cells/well in DMEM plus 1% foetal calf serum, 1% L-glutamine in 96-well plates (Costar, 3904) and incubated at 37° C. (+5% CO₂) in a humidified incubator overnight (24 hours). The following day, compounds from dry weight samples were dissolved in 100% DMSO to give 10 mM concentration. 40 μL of the compound solution was dispensed into the wells of each quadrant across the 384 Labcyte plate (Labcyte Catalogue No. P-05525) (inclusive of a positive control (100% DMSO), a negative control (10 μM) and a reference compound (250 nM)—1-tert-butyl-3-[2-{[3-(diethylamino)propyl]amino}-6-(3,5-dimethoxyphenyl)pyrido[2,3-d]pyrimidin-7-yl]urea—PD173074—a commercially available FGFR inhibitor). The 384 Labcyte plate was then transferred to the Hydra to dilute the compounds 1:100 into the remaining wells of the quadrant. 70 μL of media was aspirated from the assay plate using the Quadra before the plate was transferred onto the ECHO 550. The 384 Labcyte compound plate was also transferred onto the ECHO 550. Compound transfer to the assay plate on the ECHO 550 was at concentration ranges (1) 10 μM, (2) 3 μM, (3) 1 μM, (4) 0.3 μM, (5) 0.1 μM and (6) 0.01 μM. The plates were gently tapped to mix compound in with the cell media and left to incubate at 37° C. with 5% CO₂ for 1 hour.

Media was removed from the wells using vacuum aspiration; cells were fixed by adding 50 μL of 100% methanol to each well and incubated at RT for 20 minutes. The fixative solution was then removed and the wells were washed once with 200 μL phosphate buffered saline (PBS/A) before permeabilising the cells by the addition of 50 μL/well 0.1% triton/PBS/A for 20 minutes at RT. The permeabilisation solution was then removed and the cells washed once more with 200 μL/well PBS/A before the addition of 40 μL 1/1000 primary antibody solution (Cell Signalling Technologies #CS3476; mouse anti-phospho FGFR1 diluted in PBS/A with 10% FCS+0.1% Tween20) to each well. Following incubation at RT for 1 hour, the antibody solution was removed and the wells were washed once with 200 μL/well PBS/A. Then 40 μL 1/500 secondary antibody (A11005; goat anti-mouse 594) solution and 1/10000 Hoechst (diluted together in PBS/A with 10% FCS+0.1% Tween 20) were added and the plate incubated in the dark at RT for 1 hour. Finally, the plates were washed once with 200 μL/well PBS/A, leaving the final wash in the wells before sealing the plates. The plates were read on an Arrayscan (Cellomics). The Channel 2 (594 nm) values obtained from undosed (max) and reference compound (min) wells within a plate are used to set boundaries for 0% and 100% compound inhibition. Compound data was normalized against these values to determine the dilution range of a test compound that gives 50% inhibition of phosphorylated FGFR3.

### Cell FGFR4 (ECHO) Assay

Cell based inhibition of transiently expressed FGFR4 phosphorylation via use of ECHO technology (measured using phospho-specific primary and fluorescent secondary antibodies).

This assay is designed to detect inhibitors of transiently expressed FGFR4 phosphorylation by antibody staining of fixed cells detected using ArrayScan technology. Cos-1 cells were routinely passaged in DMEM (Gibco BRL, 41966) plus 3% foetal calf serum (FCS), 1% L-glutamine (Gibco BRL, 25030) to a confluence of 80%. To undertake the assay, Cos-1 cells were harvested at 90-95% confluence for cell transfection. For each 96-well plate, 24 μL Lipofectamine 2000 was added to 809 μL OptiMEM and incubated at room temperature for 5 minutes. For each 96 well plate, 20 μg 3′ FLAG tagged FGFR4/pcDNA3.1 (MSD 6273) was diluted with OptiMEM to a total volume of 833 μL. Equal volumes of DNA and Lipofectamine 2000 were combined (DNA:Lipid=1:1.2 ratio) and incubated at room temperature for 20 minutes.

The harvested Cos-1 cells are counted using a coulter counter and diluted further with 1% FCS/DMEM to 1.2×10⁵ cells/mL. For each 96-well, 8.33 mL cells were required. The complexed transfection solution was added to the cell solution and the cells were seeded at 1.0×10⁴ cells/well in DMEM plus 1% foetal calf serum, 1% L-glutamine in 96-well plates (Biocoat #6640) and incubated at 37° C. (+5% CO₂) in a humidified incubator overnight (24 hours).

The following day, compounds from dry weight samples were dissolved in 100% DMSO to give 10 mM concentration. 40 μL of the compound solution was dispensed into the wells of each quadrant across the 384 Labcyte plate (Labcyte Catalogue No. P-05525) (inclusive of a positive control (100% DMSO), a negative control (10 μM) and a reference compound (250 nM)). The 384 Labcyte plate was then transferred to the Hydra to dilute the compounds 1:100 into the remaining wells of the quadrant. 70 μL of media was aspirated from the assay plate using the Quadra before the plate was transferred onto the ECHO 550. The 384 Labcyte compound plate was also transferred onto the ECHO 550. Compound transfer to the assay plate on the ECHO 550 was at concentration ranges (1) 10 μM, (2) 3 μM, (3) 1 μM, (4) 0.5 μM, (5) 0.1 μM, (6) 0.03 μM (7) 0.01 μM, (8) 0.001 μM.

The plates were gently tapped to mix compound in with the cell media and left to incubate at 37° C. with 5% CO₂ for 1 hour.

Media was removed from the wells using vacuum aspiration; cells were fixed by adding 50 μL of 100% methanol to each well and incubated at room temperature for 20 minutes. The fixative solution was then removed and the wells were washed once with 200 μL phosphate buffered saline (PBS/A) before permeabilising the cells by the addition of 50 μL/well 0.1% triton/PBS/A for 20 minutes at room temperature. The permeabilisation solution was then removed and the cells washed 4 times with 100 μL/well PBS/A before the addition of 40 μL 1/1000 primary antibody solution (Cell Signalling Technologies #CS3476; mouse anti-phospho FGFR1 diluted in PBS/A with 10% FCS+0.1% Tween20) to each well. Following incubation at room temperature for 1 hour, the antibody solution was removed and the wells were washed 4 times with 100 μL/well PBS/A. Then 40 μL 1/500 secondary antibody (A11005; goat anti-mouse 594) solution and 1/10000 Hoechst (diluted together in PBS/A with 10% FCS+0.1% Tween 20) were added and the plate incubated in the dark at room temperature for one hour. Finally, the plates were washed 4 times with 100 μL/well PBS/A, and then PBS/A 200 μL/well was added before sealing the plates. The plates were read on an Arrayscan (Cellomics). The Channel 2 (594 nm) values obtained from undosed (max) and reference compound (min) wells within a plate are used to set boundaries for 0% and 100% compound inhibition. Compound data was normalized against these values to determine the dilution range of a test compound that gives 50% inhibition of phosphorylated FGFR4.

Example 1b—IC₅₀ 0.029 μM, 0.033 μM, 0.045 μM, 0.028 μM.

### Cytochrome P450 Inhibition Assay

The inhibitory potential (IC₅₀) of test compounds against 5 human cytochrome P450 (CYP) isoforms (1A2, 2C9, 2C19, 3A4 and 2D6) was assessed using an automated fluorescent end point in vitro assay modified from Crespi (Crespi and Stresser, J Pharmacol Toxicol Methods 2000, 44: 325-331). Microsomal subcellular fractions prepared from Yeast cell lines expressing each human CYP isoform were used as an enzyme source in this assay. The activity of the 5 major human CYPs was determined from the biotransformation of a number of coumarin substrates to fluorescent metabolites, in the presence of NADPH. Inhibition of these CYPs resulted in a decrease in the amount of fluorescent metabolite formed. Comparison of the fluorescence observed in the presence of varying concentrations of test compound with that seen in its absence allowed an IC₅₀ value to be calculated. Initial experiments were performed to optimise the kinetic parameters of the assay and these have been listed in Table 1. Stock solutions of each CYP, with its respective substrate, were prepared in phosphate buffer pH 7.4 (see Table 1) and 178 μl was added to the well of a black solid, flat bottom, 300 μl 96 well microtitre plate (Corning Costar). Test compounds were serially diluted in DMSO/acetonitrile and added (2 μl) to the reaction to give final concentrations of 0.1, 0.3, 1, 3 and 10 μM. After pre-incubating at 37° C. for 5 min the reactions were started with addition of NADPH (20 μl, concentration shown in Table 1). The final solvent content in each incubation was <=2% (1% from the test compound and a maximum of 1% from the substrate). The appropriate solvent controls and substrate blanks were included in each experiment to assess control activity and identify any inherent fluorescence due to the test compounds. In addition, known inhibitors of each CYP were included as positive controls (see Table 3 for inhibitor concentrations and expected IC₅₀ ranges). The reactions were stopped at defined timepoints (see Table 1) by quenching with 100 μl of solvent (acetonitrile:0.5M Tris buffer 80:20 v/v). The plates were read on a fluorimeter (Spectrafluor Plus) at the appropriate excitation and emission wavelengths (listed in Table 2) and the percent activity, corrected for control, was plotted against the test compound concentration. The IC₅₀ (the concentration of test compound required to cause 50% inhibition of metabolic activity) for each CYP was then determined from the slope of these plots.

TABLE 1
Concentrations of assay reagents and assay conditions.

| | CYP | | | Phos- | | Incu- |
|---|---|---|---|---|---|---|
| | solution | | Sub- | phate | | bation |
| | (pmol/ | | strate | Buffer | NADPH | time |
| CYP | 200 μl) | Substrate | (uM) | (M) | (μM) | (min) |
| 1A2 | 1 | 3-cyano-7- | 3 | 0.1 | 250 | 20 |
| | | ethoxy-coumarin | | | | |
| | | (CEC) | | | | |
| 2C9 | 3 | 7-methoxy-4- | 50 | 0.025 | 250 | 40 |
| | | trifluoromethyl- | | | | |
| | | coumarin (MFC) | | | | |
| 2C19 | 5 | 7-methoxy-4- | 50 | 0.05 | 250 | 60 |
| | | trifluoromethyl- | | | | |
| | | coumarin (MFC) | | | | |
| 2D6 | 3 | 7-methoxy-4- | 20 | 0.1 | 60 | 35 |
| | | (aminomethyl)- | | | | |
| | | coumarin | | | | |
| | | (MAMC) | | | | |
| 3A4 | 5 | 7-benzyloxy-4- | 15 | 0.1 | 250 | 35 |
| | | (trifluoromethyl)- | | | | |
| | | coumarin (BFC) | | | | |

TABLE 2
Excitation and emission wavelengths used by Spectrafluor Plus
Fluorimeter to detect fluorometric metabolites.

| | | | Excitation | Emission |
|---|---|---|---|---|
| CYP | Substrate | Metabolite | λ (nm) | λ (nm) |
| 1A2 | 3-cyano-7-ethoxy- | 3-cyano-7- | 405 | 460 |
| | coumarin (CEC) | hydroxy- | | |
| | | coumarin (CHC) | | |
| 2C9 | 7-methoxy-4- | 7-hydroxy-4- | 405 | 535 |
| | trifluoromethyl- | trifluoromethyl- | | |
| | coumarin (MFC) | coumarin (HFC) | | |
| 2C19 | 7-methoxy-4- | 7-hydroxy-4- | 405 | 535 |
| | trifluoromethyl- | trifluoromethyl- | | |
| | coumarin (MFC) | coumarin (HFC) | | |
| 2D6 | 7-methoxy-4- | 7-hydroxy-4- | 390 | 460 |
| | (aminomethyl)- | (aminomethyl)- | | |
| | coumarin (MAMC) | coumarin | | |
| | | (HAMC) | | |
| 3A4 | 7-benzyloxy-4- | 7-hydroxy-4- | 405 | 535 |
| | (trifluoromethyl)- | trifluoromethyl- | | |
| | coumarin (BFC) | coumarin (HFC) | | |

CEC and HFC were obtained from Ultrafine Chemicals;
CHC was obtained from Molecular Probes;
MFC, MAMC, HAMC and BFC were obtained from Gentest Corporation.

TABLE 3
Known inhibitors and optimised experimental conditions
for each of the 5 human CYP isoforms.

| | Substrate | Range of standard inhibitor | IC₅₀ range |
|---|---|---|---|
| CYP | (μM) | concentrations (μM) | (μM) |
| 1A2 | 3 | Fluvoxamine | 0.01-0.07 |
| | | 1, 0.3, 0.1, 0.03, 0.01 | |
| 2C9 | 50 | Sulphaphenazole | 0.1-1.0 |
| | | 10, 3, 1, 0.3, 0.1 | |
| 2C19 | 50 | Omeprazole | 1.5-4.6 |
| | | 10, 3, 1, 0.3, 0.1 | |
| 2D6 | 20 | Quinidine | 0.003-0.03 |
| | | 0.1, 0.03, 0.01, 0.003, 0.001 | |
| 3A4 | 15 | Ketoconazole | 0.005-0.015 |
| | | 0.25, 0.075, 0.025, 0.0075, 0.0025 | |

Fluvoxamine was obtained from Tocris Cookson Ltd;
Sulphaphenazole and Quinidine were obtained from Sigma;
Omeprazole was obtained from AstraZeneca;
Ketoconazole was obtained from Ultrafine Chemicals.

### Results

| | Ic50 | Ic50 | Ic50 | Ic50 | Ic50 |
|---|---|---|---|---|---|
| | 1A2 | 2C9 | 2C19 | 2D6 | 3A4 |
| Example 1a | >10 | 3.76 | >10 | >10 | 2.94 |
| Example 1b | >10 | 9.12 | 8.01 | >10 | >10 |

Conclusion: Enatiomers Compounds of the present invention while showing good FGFR inhibition, also show a difference in their Cytochrome P450 inhibition. Low inhibition of Cytochrome P450 is desirable to ameliorate potential drug:drug interactions.

### Physical Property Tests and Methods

### Protein Binding

Protein binding is determined by equilibrium dialysis. A 20 μM concentration of compound is dialyzed against 10% plasma at a temperature of 37° C. for 18 h. The resulting samples are analyzed using generic HPLC-UV methodology coupled with mass spectral peak identification. The reported K1 value is the first apparent association constant [proteináligand]/([protein][ligand]), all concentrations being measured in moles/liter (J. Med. Chem., 2006, 49(23), 6672-6682).

Protein binding can be measured in a high-throughput screen by equilibrium dialysis combined with liquid chromatography and mass spectrometry (Wan, H. and Rehngren, M., J. Chromatogr. A 2006, 1102, 125-134).

Example 1a: 0.91% free (rat)

Example 1b: 0.62% free (rat)

Example 1a: 3.72% free (human)

Example 1b: 2.79% free (human)

Conclusion: A reduction in protein binding indicates that there is more free drug (unbound). This may be advantageous as there may be more drug available to act at the target site.

### Clearance:

For the rat dose of 0.927 mg/kg (2 umol/kg), the compounds were formulated in 20% DMA:80% sorensens buffer pH 5 at 1 umol/ml. Each formulation was dosed (2 mL/kg) to four male rats (250-300 g) which had free access to food. Blood samples were taken via the tail vein at 5 and 20 minutes, and 1 and 4 hours post dose from 2 rats, and at 10 and 40 minutes, and 2 and 6 hours from the other 2 rats. Terminal samples were taken at 12 hours from the first pair of rats and 24 hours from the second pair. The blood samples were diluted 1:1 with water prior to analysis.

For the dog dose of 0.927 mg/kg (2 umol/kg), the compounds were formulated in 10% DMSO:90% hydroxyl-propyl-β-cyclodextrin (25% w/v) in sorensens buffer pH 5 at 2 umol/ml. Each formulation was dosed (1 mL/kg) to a male and female dog (8-15 kg) which had been fasted overnight. Blood samples were taken via the jugular vein at 5, 10, 20 and 40 minutes, and 1, 2, 4, 6, 12 and 24 hours post dose. The blood samples were diluted 1:1 with water prior to anaylsis.

A set of 10 calibration standards covering the concentration range (0.001 umol/L to 10 umol/L) were prepared by spiking blank matrix (1:1 blood:water). The samples and standards were extracted using a solid phase extraction plate, blown down under nitrogen, and then reconstituted in methanol:water (20:80). The samples were analysed using LC-MSMS and the results obtained were used to determine the area under the curve from time 0 to infinity [AUC0-inf (ug.hr/ml)], clearance [Cl (ml/min/kg)] and the steady state volume distribution [Vss (L/kg)] for each compound.

### Data for Male Rats

| | dose | Cl | Vss | AUC 0-inf |
|---|---|---|---|---|
| Example | mg/kg | (ml/min/kg) | (L/Kg) | (ug · hr/ml) |
| 1a | 0.927 | 60.9 | 2.58 | 0.254 |
| 1b | 0.927 | 32.7 | 1.74 | 0.473 |

### Data for Male Dogs

| | dose | | Vss | AUC 0-inf |
|---|---|---|---|---|
| Ex | mg/kg | Cl (ml/min/kg) | (L/Kg) | ug · hr/ml |
| 1a | 0.927 | 26.4 | 3.71 | 0.585 |
| 1b | 0.927 | 12.5 | 6.55 | 1.24 |

### Data for Female Dogs

| | dose | Cl | Vss | AUC 0-inf |
|---|---|---|---|---|
| Ex | mg/kg | (ml/min/kg) | (L/Kg) | ug · hr/ml |
| 1a | 0.927 | 36.2 | 7.08 | 0.427 |
| 1b | 0.927 | 11.5 | 1.71 | 1.35 |

Conclusion: A reduction in clearance indicates that the drug is retained for longer. This may be advantageous as it may be possible to achieve and maintain the drug exposure levels required for efficacy from smaller drug doses.

1. A compound of formula (Ia) or (Ib): 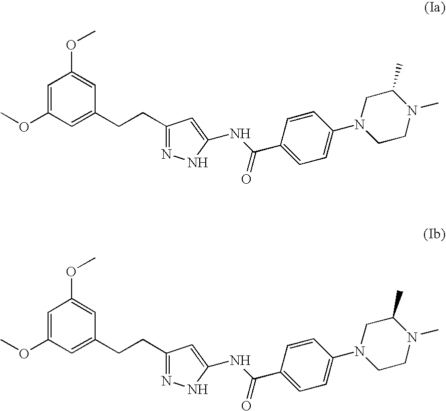
or a pharmaceutically acceptable salt thereof.

2. A compound according to claim 1 wherein the compound is a compound of formula (Ib): 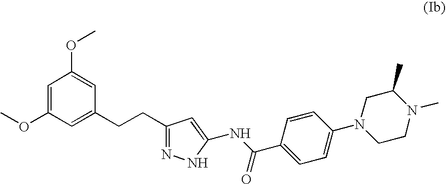
or a pharmaceutically acceptable salt thereof.

3. A compound of formula (Ib) as claimed in claim 2, or a pharmaceutically acceptable salt thereof, for use as a medicament.

5. A method for producing a FGFR inhibitory effect in a warm-blooded animal, such as man, in need of such treatment which comprises administering to said animal an effective amount of a compound of formula (Ib) as claimed in claim 2, or a pharmaceutically acceptable salt thereof.

6. A method for producing an anti-cancer effect in a warm-blooded animal, such as man, in need of such treatment which comprises administering to said animal an effective amount of a compound of formula (Ib) as claimed in claim 2, or a pharmaceutically acceptable salt thereof.

7. A pharmaceutical composition comprising a compound of formula (Ib) as claimed in claim 2, or a pharmaceutically acceptable salt thereof, as herein defined, in association with a pharmaceutically acceptable adjuvant, diluent or carrier.

8. A method of treating melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (Ib) as claimed in claim 2 or a pharmaceutically acceptable salt thereof as defined herein.

9. A pharmaceutical composition as claimed in claim 7 wherein the compound of formula (1b) is in crystalline form.

10. A pharmaceutical composition as claimed in claim 7 wherein the compound of formula (1b) is in crystalline form, referred to as Form A.
